(19) United States
(12) Patent Application Publication
(10) Pub. No.: US 2021/0380630 A1
(43) Pub. Date: Dec. 9, 2021

(54) Bio-catalyzed Synthesis of New Aromatase Inhibitors through Structural Modifications of Anticancer Drug Formestane

(71) Applicants: Atia-tul- Wahab, Karachi (PK); Muhammad Iqbal Choudhary, Karachi (PK); Rabia Farooq, Karachi (PK); Nimra Naveed Shaikh, Karachi (PK); Atta-ur- Rahman, Karachi (PK)

(72) Inventors: Atia-tul- Wahab, Karachi (PK); Muhammad Iqbal Choudhary, Karachi (PK); Rabia Farooq, Karachi (PK); Nimra Naveed Shaikh, Karachi (PK); Atta-ur- Rahman, Karachi (PK)

(21) Appl. No.: 17/403,899

(22) Filed: Aug. 17, 2021

(51) Int. Cl. C07J 1/00

(52) U.S. Cl. CPC C07J 1/0011

(57) ABSTRACT

The present invention identifies new steroidal analogues of formestane synthesized through biotransformation with significant aromatase inhibitory activity, and thus can serve as possible safe, effective, and selective anti-cancer agent towards estrogen-responsive (ER+) breast cancer. Four new derivatives of anticancer drug formestane (1), 4α,5α,7α-trihydroxyandrostane-3,17-dione (2), 3β,7β-dihydroxyandrostane-4,17-dione (3), 3β,5α,7β-trihydroxyandrostane-4,17-dione (4), and 4,11α-dihydroxyandrosta-1,4-diene-3,17-dione (5) were synthesized through bio-catalyzed structural modifications by Cunninghamella. blakesleeana and Fusarium lini. The new derivatives showed varying degree of inhibition of aromatase enzyme. Metabolite 4 (IC₅₀=0.386±0.072 μM), showed a significant inhibitory potential, comparable to substrate 1 (IC₅₀=0.335±0.011 μM) and standard aromatase inhibitory anti-cancer drug exemestane (IC₅₀=0.232±0.031 μM). Metabolites 3 (IC₅₀=1.37±0.029 μM), and 2 (IC₅₀=5.229±0.094 μM) showed significant inhibition, while metabolite 5 (IC₅₀=34.27±0.532 μM) showed a weak inhibitory activity as compared to standard.

### BACKGROUND OF THE INVENTION

Cancer is one of the major health problems and second leading cause of death globally [Siegel et al., CA-Cancer. J. Clin. 2020, 70, 7]. Breast cancer is the most common and prevalent cancer affecting approximately 2.3 million women worldwide [GLOBOCAN, 2020]. Mortality due to breast cancer is on rise, both in developed and developing countries. It is reported that in Asia, Pakistan has the highest rate of breast cancer mortality and morbidity with 90,000 cases reported annually and more than 40,000 deaths [Malik et al., J. Pak. Med. Assoc. 2019, 69, 976; Menhas et al., Iran. J. Public. Health. 2015, 44, 586; Ghoncheh et al., Asian. Pac. J. Cancer. Prev. 2015, 16, 6081]. 70-80% of all the breast cancer are hormone dependent, either require estrogen or progesterone to grow [Vasiliou and Diamandis, Crit. Rev. Clin. Lab. Sci. 2019, 56, 200; Joe, 2019]. Among all the hormone receptor breast cancers, 80% are estrogen positive. Aromatase also known as estrogen synthetase, a member of cytochrome P450 family is an important enzyme which catalyzes crucial step in the conversion of androgen to estrogen [Khan et al., Reprod. Bio. Endocrinol. 2011, 9, 1; Brueggemeier et al., Endocr. Rev. 2005, 26, 331].

A list of known human carcinogens also includes estrogens [IARC, 1987, 7, 106; Schneider et al., Climacteric. 2005, 8, 311] because elevated serum levels of estrogens have been implicated in the development of breast cancer through receptor mediated process by stimulating cell growth and proliferation [Schneider et al., Breast Cancer: Targets. Ther. 2011, 3, 113]. Therefore, mainstay of treatment for hormone receptor breast cancers is the inhibition of estrogen action which could be achieved either by blocking estrogen receptor (ER) binding to its ligand or by disrupting estrogen production. The production of endogenous estrogens reduced greatly by the inhibition of the aromatase enzyme. Therefore, aromatase enzyme is an important target to slow down the progression of hormone-responsive breast cancer.

Steroids are important class of compounds with over 300 steroidal drugs known with a broad spectrum of biological activities, such as anti-inflammatory [Siddiqui et al., J. Adv. Rev. 2020, 24, 69; Ko et al., Steroids. 2000, 65, 210], immunosuppressants, anabolic, contraceptive, diuretic, anticancer [Sultana. N, Steroids. 2018, 136, 76; Choudhary et al., Front. Pharmacol. 2017, 8, 900; Siemes et al., Steroids. 2010, 75, 1024; Fragkaki et al., Steroids. 2009, 74, 172; Tuba et al., Steroids. 2000, 65, 266] cardiovascular [Craigie et al., 2009], antifungal [Chung et al., Tetrahedron. 1998, 54, 15899], anti-obesity [Suzuki et al., U.S. Pat. No. 5,846,962, 1998], anti-HIV etc. Some steroidal glycosides were reported with antiviral activity against the Herpes simplex virus type-I [Athan et al., Phytochemistry, 2002, 59, 459]. Derivatization of available drugs or biologically active compounds by chemical methods involve the use of expensive and toxic chemicals, and catalysts, as well as many steps of protection and deprotection, as compared to biotransformation, which is an effective, and robust method to synthesize structural analogues of the parent compound.

Formestane (1), a synthetic steroid based on the structure of androstenedione (natural substrate), is used as selective aromatase inhibitor in advanced breast cancer postmenopausal women [Lonning et al., J. Steroid. Biochem. Mol. Biol. 2001, 77, 39]. Formestane also known as suicidal inhibitor of aromatase which suppresses the production of estrogen. It has been used in other countries but was never approved by the U.S.-FDA [Schneider et al., Breast Cancer: Targets. Ther. 2011, 3, 113]. Formestane (1) is orally inactive, and thus administered as intramuscular injection which is its main drawback.

### BRIEF SUMMARY OF THE INVENTION

In continuation of our research on the discovery of therapeutic agents we synthesized a series of formestane derivatives through biotransformation. Formestane (1) was incubated with Cunninghamella blakesleeana and Fusarium lini at 25±02° C. This yielded four new transformed products 2-5.

The present invention involves the testing of formestane derivatives for aromatase activity based on human placental aromatase assay. Compounds 3 (IC₅₀=1.37±0.029 μM), and 4 (IC₅₀=0.386±0.072 μM) were found to be most significant aromatase inhibitor among all transformed metabolites, as compared to the substrate 1 (IC₅₀=0.335±0.011 μM), and standard drug exemestane (IC₅₀=0.232±0.031 μM). Transformed derivatives were found to be non-cytotoxic against BJ normal cell line.

Another aspect of this invention is inhibiting aromatase enzyme to treat breast cancer in need thereof comprising administering an effective amount of formestane derivatives thereof.

### BRIEF DESCRIPTION OF DRAWINGS

FIG. 1 illustrates the structures of formestane (1) and its new metabolites, 4α,5α,7α-trihydroxyandrostane-3,17-dione (2), 3β,7β-dihydroxyandrostane-4,17-dione (3), 3β,5α,7β-trihydroxyandrostane-4,17-dione (4) via Cunninghamella blakesleeana ATCC 8688a) mediated biotransformation of drug 1, along with their aromatase inhibition. All compounds were found to be non-cytotoxic to human fibroblast (BJ) normal cell line.

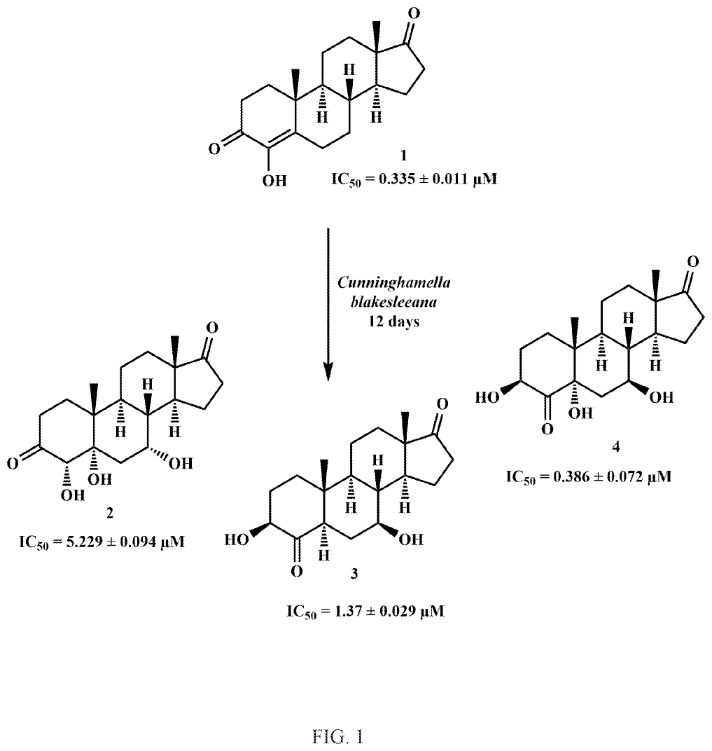

FIG. 2 illustrates the structures of formestane (1), and its new metabolite, 4,11α-dihydroxyandrosta-1,4-diene-3,17-dione (5) via Fusarium lini (NRRL 2204) mediated biotransformation of drug 1, along with their aromatase inhibition. Compound 5 was found to be non-cytotoxic to human fibroblast (BJ) normal cell line.

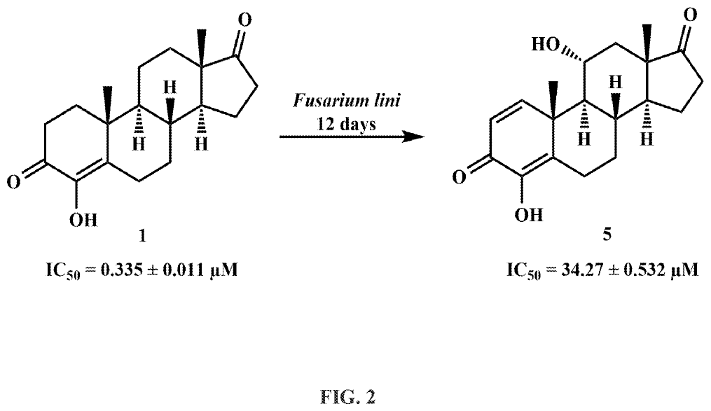

### DETAILED DESCRIPTION OF THE INVENTION

### General Experimental Protocol for Biotransformation of Formestane (1)

Formestane (1), (m/z=302.4, C₁₉H₂₆O₃), procured from Bettersyn and Co. Ltd (China). Media constituents were acquired from Sigma-Aldrich (Germany), Dae-Jung Chemicals and Metals Co., Ltd. (Korea), and Oxoid Ltd. (England). Degree of transformations and the purity of compounds 1-6 were analyzed by using silica coated thin layer chromatographic (TLC) plates (PF₂₅₄, Merck KGaA, Germany). The brown color gummy crude was fractionated by using column chromatography (CC) (Silica gel, 70-230 mesh, E. Merck, Germany). Fractions were fully purified via recycling reverse phase HPLC (LC-908, YMC L-80) 20-250 mm i.d. 4-5 μm using methanol/water. Optical rotations, IR absorbances, and melting points of all purified compounds 2-5 were measured on JASCO P-2000 polarimeter (Japan), Bruker Vector 22 spectrophotometer (France), and Büchi 560 device (Switzerland), respectively. ¹H- (400 and 500 MHz), ¹³C- (100 and 125 MHz) and 2D-NMR spectra were recorded in CD₃OD on Bruker Avance-NMR spectrometer (Switzerland). EI- and HR-EIMS, of all purified compounds 2-5 were determined on Jeol JMS-600H mass spectrometer (Japan). All the solvents used in this research work were of analytical grade.

### Fungal Cell Cultures

Fungal cell cultures Cunninghamella blakesleeana, and Fusarium lini were purchased from ATCC (American Type Culture Collection), and NRRL (Northern Regional Research Laboratories) respectively. C. blakesleeana ATCC 8688A, and F. lini NRRL were cultured on SDA slants (Sabouraud Dextrose Agar) and maintain at 4° C.

### Media Preparation

Six liters aqueous media was prepared for the culture growth of Cunninghamella blakesleeana as follows: Glucose (10.0 g), peptone (5.0 g), yeast extract (5.0 g), KH₂PO₄ (5.0 g), NaCl (5.0 g), and glycerol (10.0 mL) were added per liter of the distilled water. 300 mL of aqueous media was added into each flask of 1 L. For sterilization flasks were cotton plugged, autoclaved, and cooled. In the same way three liters of media was prepared for the culture growth of Fusarium lini NRRL 2204.

### Fermentation of Drug

Based on the small screening results media was prepared and under sterilized conditions, C. blakesleeana was inoculated into each flask containing sterilized media. Flasks were labelled and kept at 25±02° C. on rotary shaker for three days for culture growth. After appropriate growth, each cultured flask was fed with formestane (1) (800 mg of drug 1 was dissolved in 20 mL of acetone). Flasks were then incubated at 25±02° C. for twelve-days on a rotary-shaker.

Flask cultured with F. lini were fed with formestane (1) (300 mg of drug 1 was dissolved in 3 mL of acetone), and then incubated in the same way for twelve-days on a shaker.

### Extraction of Metabolites

After twelve days of incubation, reaction was stopped by adding dichloromethane into each flask. Flasks were filtered, and extracted with CH₂Cl₂. Organic layer was evaporated using high vacuum, yielding a gummy crude material.

### Purification of Metabolites

The gummy crude material was fractionated through silica gel column chromatography by using acetone-hexanes mixture with 5% gradient system. Metabolites were further purified by using recycling reverse phase high performance liquid chromatography (LC-908W, YMC L-80 (4-5 μm, 20×250 mm i.d.). Metabolites 2 (H₂O/CH₃OH, 3/7; Rₜ=34 min, 15 mg), 3 (H₂O/CH₃OH, 4/6; Rₜ=18 min, 7 mg), 4 (H₂O/CH₃OH, 5/5; Rₜ=34 min, 12 mg), and 5 (H₂O/CH₃OH, 3/7; Rₜ=20 min, 10 mg) were purified via recycling reverse HPLC from fraction 1-3 from B. bassiana, and a fraction from F. lini, respectively.

### 4α,5α,7α-Trihydroxyandrostane-3,17-dione (2)

White solid; melting point: 220-221° C.; [α]_{D}²⁵=−56 (c 0.003, CH₃OH); IR υₘₐₓ (cm⁻¹): 3371 (O—H), 2927 (C—H stretching), 1730 (C═O); EI-MS m/z (%): 336 [M]⁺ (5); HREI-MS m/z); 336.19548 [M]⁺ (calc. 336.1937 (mol. formula C₁₉H₂₈O₅); ¹H-NMR (δ) (d₄-methanol), H₂-1 (2.05, m, 1.50 m), H₂-2 (2.17, m, 1.56 overlap), H-4 (4.78, overlap), H₂-6 (1.95, dd, J=14.8, 2.8 Hz, 1.77, overlap), H-7 (4.07, d, J=2.8 Hz), H-8 (1.71, overlap), H-9 (1.90, td, J=12.0, 4.4 Hz), H₂-11 (1.67, overlap, 1.40, td, J=12.8, 3.6 Hz), H₂-12 (1.78, overlap, 1.28, td, J=13.2, 4.0 Hz), H-14 (1.79, overlap), H₂-15 (2.03, overlap, 1.60, m), H₂-16 (2.45, dd, J=18.8, 9.2 Hz, 2.07, m), H₃-18 (0.87, s), H₃-19 (0.78, s); ¹³C-NMR (δ) (d₄-methanol), C-1 (30.5), C-2 (33.1), C-3 (212.6), C-4 (72.0), C-5 (82.9), C-6 (32.7), C-7 (67.9), C-8 (39.7), C-9 (40.6), C-10 (45.9), C-11 (21.7), C-12 (32.6), C-13 (46.0), C-14 (46.9), C-15 (22.0), C-16 (36.5), C-17 (223.6), C-18 (14.0), C-19 (15.2).

### 3β,7β-Dihydroxyandrostane-4,17-dione (3)

White solid; melting point: 200-203° C.; [α]_{D}²⁵=+61 (c 0.0008, CH₃OH); IR υₘₐₓ (cm⁻¹): 3399 (O—H), 2943 (C—H stretching), 1723 (C═O); EI-MS m/z (%): 320 [M]⁺ (68); HREI-MS m/z); 320.1984 [M]⁺ (calc. 320.1988 (mol. formula C₁₉H₂₈O₄); ¹H-NMR (δ) (d₄-methanol), H₂-1 (1.90, m, 1.54, overlap), H₂-2 (2.24, overlap, 1.65, ddd, J=17.5, 14.5, 4.0 Hz), H-3 (4.17, dd, J=11.6, 7.6 Hz), H-5 (2.36, overlap), H₂-6 (1.85, m, 1.54, overlap), H-7 (3.38, m), H-8 (1.55, overlap), H-9 (1.10, m), H₂-11 (1.77, overlap, 1.36, ddd, J=16.5, 12.5, 3.5 Hz), H₂-12 (1.78, overlap, 1.25, td, J=15.0, 3.5 Hz), H-14 (1.55, overlap), H₂-15 (2.26, overlap, 1.91, overlap), H₂-16 (2.39, overlap, 2.04, dt, J=19.5, 9.2 Hz), H₃-18 (0.87, s), H₃-19 (0.76, s); ¹³C-NMR (δ) (d₄-methanol), C-1 (36.6), C-2 (33.2), C-3 (75.8), C-4 (212.3), C-5 (55.0), C-6 (31.5), C-7 (74.5), C-8 (43.1), C-9 (53.9), C-10 (43.3), C-11 (22.2), C-12 (32.6), C-13 (49.8), C-14 (52.1), C-15 (25.9), C-16 (36.8), C-17 (224.3), C-18 (14.4), C-19 (14.1).

### 3β,5α,7β-Trihydroxyandrostane-4,17-dione (4)

White solid; melting point: 195-196° C.; [α]s=−141 (c 0.0007, CH₃OH); IR υₘₐₓ(cm⁻¹): 3396 (O—H), 2934 (C—H stretching), 1738 (C═O); EI-MS m/z (%): 336 [M]⁺ (7); HREI-MS m/z); 336.1921 [M]⁺ (calc. 336.1937 mol. Formula C₁₉H₂₈O₅); ¹H-NMR (δ) (d₄-methanol), H₂-1 (1.94, m, 1.49 dt, J=13.0, 4.0 Hz), H₂-2 (2.20, m, 1.56, overlap), H-3 (4.89, dd, J=11.5, 8.0 Hz), OH-5 (4.89 in d₆-acetone), H₂-6 (1.86, overlap, 1.79, overlap), H-7 (3.73, ddd, J=11.0, 9.5, 5.0 Hz), H-8 (1.59, overlap), H-9 (1.74, overlap), H₂-11 (1.61, overlap, 1.39, m), H₂-12 (1.75, overlap, 1.27, td, J=12.8, 4.0 Hz), H-14 (1.54, overlap), H₂-15 (2.25, overlap, 1.87, overlap), H₂-16 (2.40, dd, J=19.5, 8.5 Hz, 2.06, m), H₃-18 (0.88, s), H₃-19 (0.82, s); ¹³C-NMR (δ) (d₄-methanol), C-1 (30.4), C-2 (33.9), C-3 (72.1), C-4 (214.0), C-5 (81.4), C-6 (37.5), C-7 (71.5), C-8 (43.1), C-9 (45.8), C-10 (44.9), C-11 (22.1), C-12 (32.8), C-13 (49.7), C-14 (52.6), C-15 (25.7), C-16 (36.9), C-17 (224.3), C-18 (14.4), C-19 (15.8).

### 4,11α-Dihydroxyandrosta-1,4-diene-3,17-dione (5)

White solid; melting point: 171-174° C.; [α]=+39 (c 0.001, CHCl₃); IR υₘₐₓ (cm⁻¹): 3417 (O—H), 2925 (C—H stretching), 1737 (C═O), 1633 (C═C); UV λₘₐₓ (log ε): 248 (6.26); EI-MS m/z (%): 316 [M]⁺ (92.5); HREI-MS m/z); 316.1661 [M]⁺ (calc. 316.1675 mol. Formula C₁₉H₂₄O₄); ¹H-NMR (δ) (d₄-methanol), H-1 (7.95, d, J=10.5 Hz), H-2 (6.18, d, J=10.5 Hz), H₂-6 (3.22, m, 2.08 overlap), H₂-7 (2.06, overlap, 1.09 overlap), H-8 (1.92, overlap), H-9 (1.08, overlap), H-11 (4.04, td, 10.5, 5.0 Hz), H₂-12 (2.09, overlap, 1.21, t, J=12.0 Hz), H-14 (1.39, m), H₂-15 (1.95, overlap, 1.63 m), H₂-16 (2.46, dd, J=19.5, 8.0 Hz, 2.10, overlap), H₃-18 (0.96, s), H₃-19 (1.33, s); ¹³C-NMR (δ) (d₄-methanol), C-1 (162.9), C-2 (122.8), C-3 (182.4), C-4 (143.8), C-5 (139.5), C-6 (23.7), C-7 (32.4), C-8 (35.1), C-9 (62.3), C-10 (42.9), C-11 (68.2), C-12 (43.0), C-13 (45.5), C-14 (50.9), C-15 (22.8), C-16 (36.7), C-17 (222.0), C-18 (14.8), C-19 (19.0).

### Protocol of Human Placental Aromatase Assay

The aromatase enzyme involved in the last step of conversion of testosterone into estradiol (conversion of androgen to estrogen). Therefore, the aromatase activity can be determined by the conversion of testosterone to estradiol

After the ethical clearance (ICCBS/IEC-030-HT-2018/Protocol/1.0), the human placental aromatase assay was performed. The reaction mix containing protein (2 mg) from human placental microsomes, 10 μM testosterone (10 μL) and test sample (0.1 mM) in methanol making a total volume of 1 mL. The reaction mixture was pre-incubated at 37° C. for 10 minutes. To initiate the reaction NADPH (1 mM) was added, containing potassium phosphate (0.1 M) at pH 7.4, and incubated for 20 minutes. 100 μL of trichloroacetic acid (10%, w/v) was then added to terminate the reaction, followed by centrifugation at 12,000 g for 10 minutes, resulting pellet was discarded. The supernatant was extracted with n-butyl chloride (1 mL), and the sample was dried. The amount of 17β-estradiol in the supernatant was determined through UPLC, using ACN/H₂O (45:55, v/v with triethylamine 0.1%) as a mobile phase at pH of 3.0. The pH was adjusted by adding orthophosphoric acid. Isocratic elution (flow rate=1.2 mL/min) was carried out at 200 nm. Calculations were performed by using the following formula:

% Inhibition=100−(Peak area of test sample/Peak area of control)×100

### Results and Discussion

Fermentation of anti-cancer drug, formestane (1), with C. blakesleeana resulted into three new metabolites 2-4, and Fusarium lini yielded a new transformed product 5. Structure of the transformed products were determined with the help of spectroscopic techniques.

HREI-MS data of metabolite 2 ([M]⁺=336.1954) supported a molecular formula of C₁₉H₂₈O₅. The increase in the atomic mass units (a.m.u.), as compared to the formestane suggested the presence of two additional hydroxyl group. Comparison of ¹H- and ¹³C-NMR spectroscopic data of 1 with the 2D-NMR (¹H⁻¹H COSY, HSQC, and HMBC) data of compound 2 confirmed the hydroxylation at C-5, and C-7. OH, at C-5 was inferred through the ²J interactions of H-4, H₂-6, and ³J interactions of H₃-19, with C-5 in the heteronuclear multiple bond correlations spectrum of metabolite 2. The new downfield methine proton at δ 4.07 showed heteronuclear correlations with carbon at δ 82.9 (C-5), and 39.7 (C-8) which suggested its position at C-7. Thus, new metabolite 2 was identified as 4α,5α,7α-trihydroxyandrostane-3,17-dione.

HREI-MS of derivative 3 displayed its M⁺ at m/z 320.1984 supporting formula C₁₉H₂₈O₄. Presence of hydroxyl group in substrate 1 was determined via 1D-, and 2D-NMR spectral data. OH, at C-7 was inferred through the ²J interactions of H-8, H-6 with C-7 in the HMBC spectrum of derivative 3, as well as carbonyl moiety at C-4 within ring A. Structure of new transformed product 3 was thus deduced as 3β, 7β-dihydroxyandrostane-4,17-dione.

HREI-MS data of metabolite 4 ([M]⁺=336.1921) implied formula of C₁₉H₂₈O₅. Comparison of ¹H and ¹³C-NMR spectroscopic data of 1 with the 2D-NMR (¹H⁻¹H COSY, HSQC, and HMBC) data of compound 4 confirmed the hydroxylation C-5, and C-7, as well as carbonyl moiety at C-4 within ring A. The stereochemistry at the newly generated stereocenter C-7 was assigned as β on the basis of dipolar correlation of H-7 (δ 3.73) with α-oriented H-9 (δ 1.74), and H-14 (δ 1.54) in NOESY spectrum, which suggested β-oriented (equatorial) hydroxyl group at C-7. Structure of new transformed product 4 was thus deduced as 3β,5α,7β-trihydroxyandrostane-4,17-dione.

HREI-MS of derivative 5 displayed its M⁺ at m/z 316.1661 in agreement of formula C₁₉H₂₄O₄. Presence of hydroxyl group was determined via 1D-, and 2D-NMR spectral data. OH, at C-11 was inferred through the ²J interactions of H-9, H₂-12 with C-11 in the HMBC spectrum of metabolite 5. A new olefinic proton signal was observed for H-1 (7.95 d, J=10.5 Hz), and H-2 (6.182 d, J=10.5 Hz). The HMBC spectrum showed ^{2,3}J couplings from H-2 to C-3 (δ_{C} 144.7), C-4 and C-10. The stereochemistry at C-11 was assigned as a on the basis of dipolar correlations of β-oriented H-8 (δ 1.92), and H₃-19 (δ 1.33) with H-11 (δ 4.04) in NOESY spectrum. Structure of new transformed product 5 was thus deduced as 4,11α-dihydroxyandrosta-1,4-diene-3,17-dione.

All transformed products were subjected to human placental microsomal aromatase assay for enzyme inhibitory activity. In metabolite 2 (IC₅₀=5.229±0.094 μM), presence of α-OH at C-7, C-5 along with reduced double bond between C-4/C-5 led to decreased aromatase inhibitory activity as compared to the parent molecule 1 (IC₅₀=0.335±0.011 μM), and standard drug exemestane (IC₅₀=0.232±0.031 μM). Similarly, presence of ketonic carbonyl at C-4 and β-OH at C-7, C-3 along with the reduced olefinic moiety between C-4/C-5 in metabolite 3 (IC₅₀=1.37±0.029 μM) showed slight decrease in its aromatase inhibition potential but still is a significant inhibitor as compared to the standard drug exemestane (IC₅₀=0.232±0.031 μM). In metabolite 4 (IC₅₀=0.386±0.072 μM) reduction of olefinic moiety between C-4/C-5, and conversion of ketonic carbonyl moiety at C-3 into secondary β-OH, and presence of α-OH at C-5 and carbonyl at C-4 has not much affected its aromatase inhibition potential as compared to the parent molecule 1 (IC₅₀=0.335±0.011 μM), and standard drug exemestane (IC₅₀=0.232±0.031 μM). Presence of double bond between C-1/C-2 and α-OH at C-11 has resulted decrease in the activity of metabolite 5 (IC₅₀=34.27±0.532 μM) as compared to the parent drug. Metabolite 4 was found most active metabolite among all the transformed products.

What is claimed is:

1. A method of treatment of diseases associated with the over-expression of aromatase enzyme, including breast cancer, and male infertility, based on administration of effective amount of newly developed aromatase inhibitors having formulae 2-5 or their isomers, salts or solvates, or co-crystals in suitable pharmaceutical excipients, adjuvant, carrier, or diluent to humans, and animals in need thereof.

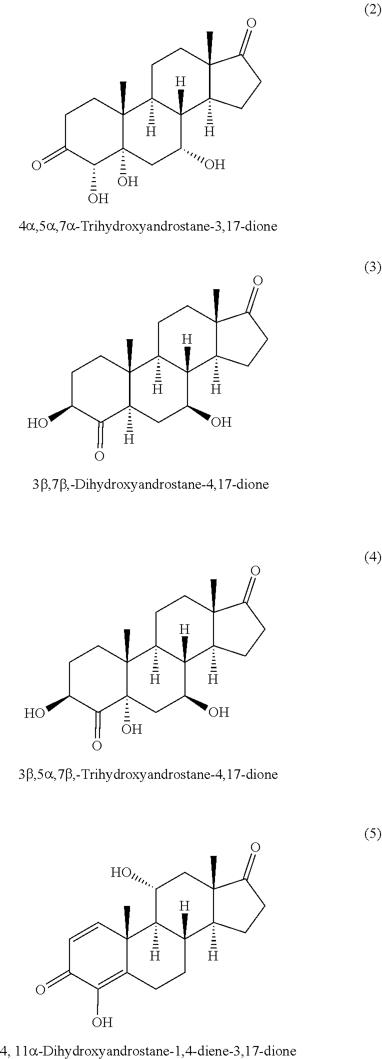

2. Formulae 2-5 as in claim 1 are new steroidal-based aromatase inhibitors that reduces, or inhibits the activity of aromatase enzyme, and thereby can treat estrogen-responsive (ER+) breast cancer, and improving testosterone/estradiol (T/E) ratio levels.

3. Formulae 2-5 can be synthesized by biotransformation of anti-cancer drug formestane (1) or through the chemical synthesis.

4. Formulae 2-5 can also be used for the prevention of other diseases resulted from the over-expression of aromatase enzyme.
